(19) United States
(12) Patent Application Publication
(10) Pub. No.: US 2020/0216483 A1
(43) Pub. Date: Jul. 9, 2020

(54) FUCOSE SEPARATION METHOD AND APPARATUS THEREFOR

(71) Applicant: ADVANCED BIOMASS R AND D CENTER, Daejeon (KR)

(72) Inventors: Yong Keun CHANG, Daejeon (KR); Sungyong MUN, Seoul (KR); Seokbin HONG, Daejeon (KR); Jae-hwan CHOI, Seoul (KR)

(21) Appl. No.: 16/605,257

(22) PCT Filed: Apr. 16, 2018
(86) PCT No.: PCT/KR2018/004394

§ 371 Date: Oct. 15, 2019

(30) Foreign Application Priority Data
Apr. 17, 2017 (KR) 10-2017-0049177

(51) Int. Cl. C07H 1/06; C07H 3/02; B01J 20/26; B01D 15/18

(52) U.S. Cl. CPC C07H 1/06; C07H 3/02; B01D 15/185; B01D 15/1828; B01D 15/1842; B01J 20/261

(57) ABSTRACT

The present invention relates to an SMB-based fucose separation method and an apparatus therefor and, more particularly, to a method and an apparatus for continuously separating fucose from a microalgae-derived monosaccharide mixture or a multi-component mixture (monosaccharide substances, amino acid substances, and glycerol components) using an SMB process.

### TECHNICAL FIELD

The present invention relates to a method of separating fucose from microalgae and an apparatus therefor.

### BACKGROUND ART

Fucose is a rare sugar belonging to the deoxy sugar family, and recently has been reported to have high industrial utility value as a raw material for use in anti-aging and hypoallergenic cosmetics, anti-cancer agents, anti-allergy agents, anti-inflammatory agents, medicine for improving long-term memory and immunity, and health functional foods (S. Hasegawa et al., J. Invest. Dermatol. 75 (1980) 284-287).

In addition, fucose is known to be useful as an artificial synthetic precursor of fucosyllactose, which is a major component of human milk oligosaccharide (HMO) (F. Baumgartner et al., Microb. Cell Fact. 12 (2013) 40). The following three methods have been reported in the literature associated with the production of fucose, which is known to have high industrial value in the future. First, fucose can be obtained by performing a chemical synthesis process (chemical configuration inversion) on monosaccharides that can be supplied in large quantities (H. Kristen et al., J. Carbohyd. Chem. 7 (1988) 277-281; G. D. Gamalevich et al., Tetrahedron 12 (1999) 3665-3674). Second, fucose can be obtained through a biological synthesis process using microorganisms (P. Vanhooren et al., J. Chem. Technol. Biotechnol. 74 (1999) 479-497; C. Wong et al., U.S. Pat. No. 6,713,287 (1995)). Third, fucose can be obtained from fucose-containing biomass present in nature (P. Saari et al., J. Liq. Chromatogr. Relat. Technol. 32 (2009) 2050-2064; A. Gori et al., EP Patent 2616547 (2011)). A typical case is a method of producing fucose through hydrolysis of hemicellulose contained in birch, beech, willow and the like.

The conventional fucose production methods mentioned above are known to have the following problems. First, the method of obtaining fucose through a chemical synthesis process is reported to have low industrial feasibility and economic efficiency due to the use of several processing steps and expensive solvents and reagents. Second, the biological synthesis method using microorganisms has low feasibility because a large-scale economic process that can efficiently isolate fucose from various byproducts (monosaccharides) produced through hydrolysis of fermentation products, that is, polysaccharide, has not been established to date. Third, the method of obtaining fucose from natural wood biomass is known to have low economic efficiency due to the problem of raw material supply cost due to the necessity of securing large quantities of fucose-containing wood, environmental damage caused by the use of natural wood, and the absence of a high-efficiency separation/purification process capable of isolating fucose from hydrolysis products of fucose-containing biomass.

Overall, when overall taking into consideration the problems associated with the conventional fucose production methods, the main obstacle to be overcome in order to realize a dramatic improvement in the economic efficiency of fucose production is to develop a process capable of isolating and purifying fucose at high purity and high efficiency from the hydrolysis products of fucose-containing monosaccharides or biomass. Also, when the cost of supplying fucose raw materials can be minimized, it is expected that the feasibility of industrialization of fucose production will ultimately be much higher. In order to realize these aspects, the following guidelines are set according to the present invention. First, a novel type of fucose separation process is developed based on a continuous separation mode having excellent economic efficiency and separation efficiency. Second, residual waste generated during the process of producing high value-added bioproducts other than fucose is used as a raw material to produce fucose. In this regard, according to the present invention, it has been recently identified through the literature that the residual waste generated after extraction of lipids (biodiesel crude oil) from microalgae (N. oceanica) can be utilized as a source of fucose raw materials (J. Park et al., Bioresour. Technol. 191 (2015) 414-419). The reason for this is that fucose is contained in the monosaccharide mixture produced after hydrolysis of this residual waste (defatted microalgal biomass). The monosaccharide components included in addition to the fucose are a total of six types of monosaccharides, namely rhamnose, ribose, xylose, mannose, glucose and galactose.

Therefore, the present invention aims to develop a process capable of continuously isolating fucose from a monosaccharide mixture derived from defatted microalgal biomass at high purity and high yield. In order to realize this aim, simulated moving-bed technology (L. S. Pais et al., AIChE J. 44 (1998) 561-569; A. G. O'Brien et al., Angew. Chem.-Int. Edit. 51 (2012) 7028-7030), the value of which is recognized in downstream processing in the biological, pharmaceutical and fine chemical industries, is introduced into the continuous fucose production process according to the present invention.

For a brief description of the simulated moving-bed (SMB) technology, a schematic diagram of the 4-zone closed loop SMB, which is a general structure of the SMB process, is shown in FIG. 1 (Z. Ma et al., AIChE J. 43 (1997) 2488-2508). As shown in FIG. 1, the SMB process consists of several columns, each of which is filled with an adsorbent having selectivity for feed mixture components. These columns are connected to one another and are divided into four zones through four ports (desorbent, extract, feed and raffinate). These four ports are moved by the length of one column along the advancement direction of a solvent at a predetermined interval (port-switching time). When the flow rate and port-switching time of the SMB process are optimal under these circumstances, the feed port can always be placed in an overlapping region (where the solute bands of two different components overlap), and the extract and raffinate ports can always be placed in a separated region (where the solute bands of two different components are separated from one another). When these circumstances are continuously maintained, continuous injection of the feed mixture and continuous recovery of each product are possible. In addition, product recovery is possible at high purity and high yield even under the circumstance of “partial separation”, in which the solute bands of two different components (fast-migrating component and slow-migrating component) are partially rather than completely separated in the SMB column (Y. Xie et al., Ind. Eng. Chem. Res. 42 (2003) 4055-4067). An SMB separation method based on this principle can secure high productivity and high separation efficiency compared to other separation methods.

Therefore, as a result of extensive efforts to develop a fucose production method capable of efficiently separating only fucose from various byproducts without using expensive solvents or reagents and preventing problems associated with raw material supply cost and environmental damage for securing large quantities of fucose-containing wood, the present inventors have developed an SMB process that is capable of continuously separating fucose from a microalgae-derived multi-component mixture and have found that fucose can be continuously separated at a high purity of 97% without causing any loss of fucose. Based on this finding, the present invention has been completed.

### DISCLOSURE OF INVENTION

It is one object of the present invention to provide a method of continuously separating fucose at high purity from a microalgae-derived multi-component mixture without causing any loss of fucose.

It is another object of the present invention to provide a device for separating fucose using the method.

In accordance with one aspect of the present invention, the above and other objects can be accomplished by the provision of a method of separating fucose based on SMB including injecting a desorbent into a desorbent port DP, recovering fucose from an extract port EP, injecting a microalgae-derived multi-component mixture into a feed port FP, and discharging other multi-component substance from a raffinate port RP, wherein fucose is separated using a porous polydivinylbenzene-based hydrophobic adsorbent in a plurality of columns connected to the respective ports.

In accordance with another aspect of the present invention, there is provided a device for separating fucose based on SMB to separate fucose including a desorbent port DP, an extract port EP, a feed port FP, a raffinate port RP, a plurality of rotary valves 10, 20, 30 and 40 selectively connected to the ports DP, EP, FP and RP, respectively, and a plurality of columns 100, 200, 300 and 400 respectively provided in the plurality of rotary valves.

### BRIEF DESCRIPTION OF THE DRAWINGS

FIG. 1 is a schematic diagram of a 4-zone closed-loop SMB process, which is a conventional typical SMB process;

FIG. 2 shows an SMB experimental apparatus according to an embodiment of the present invention;

FIG. 3 shows the result of a pulse injection experiment on polydivinylbenzene-based hydrophobic adsorbent candidate groups according to an embodiment of the present invention;

FIG. 4 shows the result of a tracer molecule pulse injection experiment performed on a finally selected adsorbent (polydivinylbenzene-based hydrophobic adsorbent having a pore size of 100 Å) according to an embodiment of the present invention;

FIG. 5 shows the result of multiple frontal analysis experiment performed on each of monosaccharide components containing fucose according to an embodiment of the present invention;

FIG. 6 shows equilibrium capacity (q*) data on the selected adsorbent of each monosaccharide component containing fucose according to an embodiment of the present invention;

FIG. 7 shows the result of a comparison between mixture frontal experiment data injecting a monosaccharide mixture according to an embodiment of the present invention as a feed and the corresponding simulation profile;

FIG. 8 shows two configuration forms suitable for optimal design of an SMB process for fucose separation according to an embodiment of the present invention;

FIG. 9 shows the result of simulation for the column profile of a periodic steady state of the SMB process for fucose separation according to an embodiment of the present invention;

FIG. 10 shows the result of a continuous separation experiment using the SMB process for fucose separation according to an embodiment of the present invention;

FIG. 11 shows HPLC analysis chromatograms of feed samples and final outlet port samples obtained in the final step regarding the SMB process experiment for fucose separation according to an embodiment of the present invention;

FIG. 12 shows a process scheme and separation sequence (Ring I SMB→Ring II SMB) suitable for additional design of the SMB process for fucose separation regarding the multi-component mixture (monosaccharide+amino acid+glycerol) according to an embodiment of the present invention;

FIG. 13 shows the result of a continuous separation experiment regarding a Ring I SMB unit of the multi-component mixture (monosaccharide+amino acid+glycerol) during the SMB process for fucose separation according to an embodiment of the present invention; and

FIG. 14 shows the result of a continuous separation experiment regarding a Ring II SMB unit of the multi-component mixture (monosaccharide+amino acid+glycerol) during the SMB process for fucose separation according to an embodiment of the present invention.

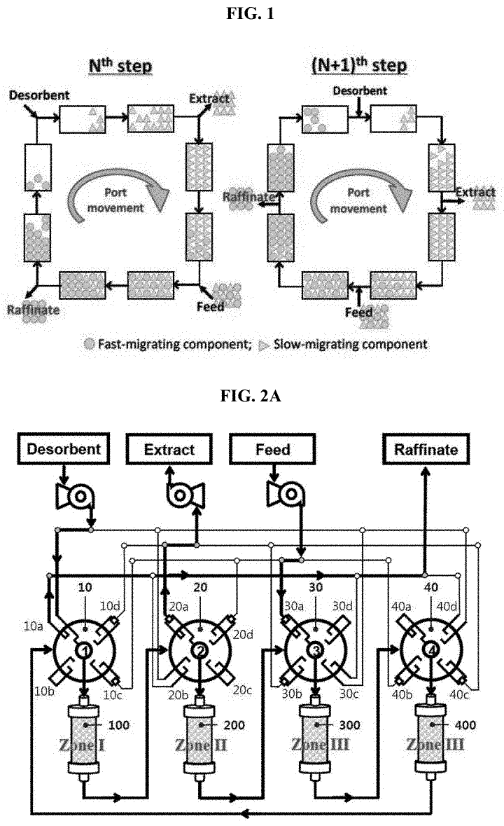

### BEST MODE FOR CARRYING OUT THE INVENTION

The present invention can be completely accomplished based on the following description. The following description should be understood to describe preferred specific examples of the invention, but the present invention is not necessarily limited thereto. In addition, the accompanying drawings are provided for better understanding, but the present invention is not limited thereto, and the details of individual configurations will be properly understood based on the specific gist of the related description given below.

Unless defined otherwise, all technical and scientific terms used herein have the same meanings as appreciated by those skilled in the field to which the present invention pertains. In general, the nomenclature used herein is well-known in the art and is ordinarily used.

According to the present invention, as a result of separating fucose from a microalgae-derived multi-component mixture using a device for separating fucose based on SMB in order to separate fucose, including a desorbent port DP, an extract port EP, a feed port FP, a raffinate port RP, a plurality of rotary valves 10, 20, 30 and 40 selectively connected to the ports DP, EP, FP and RP, respectively, and a plurality of columns 100, 200, 300, and 400 respectively provided in the plurality of rotary valves, fucose could be continuously separated at a high purity of 97% or more from a microalgae-derived multi-component mixture without causing any loss of the fucose, while using expensive solvents and reagents.

Thus, in one aspect, the present invention is directed to a method of separating fucose based on SMB including injecting a desorbent into a desorbent port DP, recovering fucose from an extract port EP, injecting a microalgae-derived multi-component mixture into a feed port FP, and discharging other multi-component substance from a raffinate port RP, wherein fucose is separated using a porous polydivinylbenzene-based hydrophobic adsorbent in a plurality of columns connected to the respective ports.

In another aspect, the present invention is directed to a device for separating fucose based on SMB using the method, including a desorbent port DP, an extract port EP, a feed port FP, a raffinate port RP, a plurality of rotary valves 10, 20, 30 and 40 selectively connected to the ports DP, EP, FP and RP, respectively, and a plurality of columns 100, 200, 300, and 400 respectively provided in the plurality of rotary valves.

In the present invention, the polydivinylbenzene-based hydrophobic adsorbent preferably has a pore size of 50 Å to 900 Å, more preferably 50 Å to 500 Å.

In the present invention, the desorbent injected into the desorbent port DP is preferably water, a buffer, an acidic solution or a basic solution.

In the present invention, the purity of the fucose recovered from the extract port EP is preferably 90% or more, more preferably 95% to 99.999%.

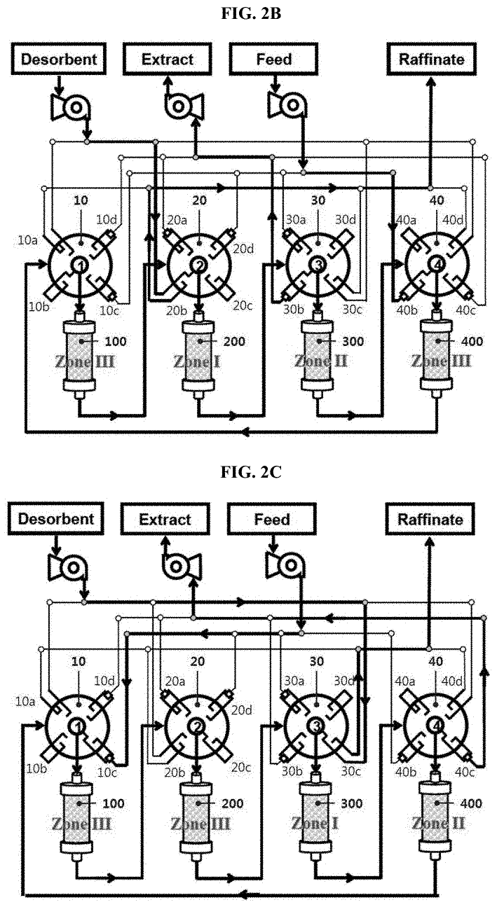

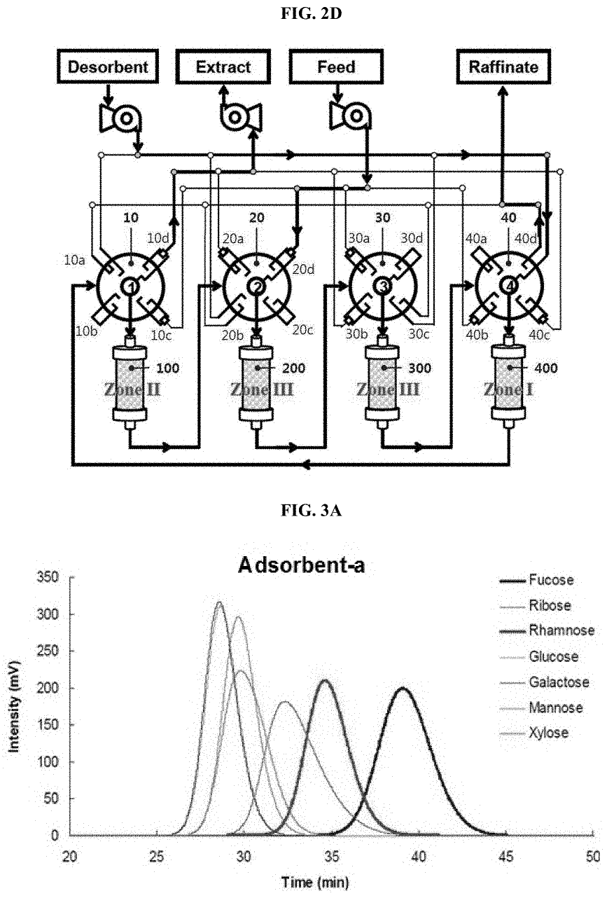

As shown in FIGS. 2A to 2D, the separation device according to the present invention includes a desorbent port DP, an extraction port (EP), a feed port FP, a raffinate port RP, a plurality of rotary valves 10, 20, 30 and 40 selectively connected to the ports DP, EP, FP and RP, respectively, and a plurality of columns 100, 200, 300 and 400 respectively provided in the plurality of rotary valves.

The four rotary valves 10, 20, 30 and 40 each have four connection ports 10a, 10b, 10c, 10d, 20a, 20b, 20c, 20d, 30a, 30b, 30c, 30d, 40a, 40b, 40c, 40d, wherein, as the rotary valve rotates, only one connection port of each rotary valve is opened, so that the connection port is in fluid communication with the desorbent port DP, the extract port EP, the feed port FP or the raffinate port RP.

In other words, each of the flow paths connected to the desorbent port DP, the extract port EP, the feed port FP and the raffinate port RP has four branch points, and is thus connected to all of four rotary valves 10, 20, 30 and 40, and is then connected a specific rotary valve as any one connection port is opened.

FIG. 2A shows a first step port position, FIG. 2B shows a second step port position, FIG. 2C shows a third step port position, and FIG. 2D shows a fourth step port position. The first to fourth step port positions are cycled continuously. That is, the device according to the present invention operates in the order of the first step port position→second step port position→third step port position→fourth step port position and then returns to the first step port position.

The setting to a particular port position is conducted by the rotation of the rotary valves 10, 20, 30, 40. That is, when the first connection ports 10a, 20a or 30a of the rotary valves 10, 20, 30 and 40 are opened, the first step port position is set, and when the rotary valves 10, 20, 30 and 40 are rotated, the second connection ports 20b, 30b and 40b are opened and the second step port position is set. When the rotary valves 10, 20, 30 and 40 are rotated again, the third connection ports 30c, 40c and 10c are opened and the third step port position is set, and when the rotary valves 10, 20, 30, and 40 are rotated again, the fourth connection ports 40d, 10d, and 20d are opened and the fourth step port position is set. Then, when the rotary valves 10, 20, 30 and 40 are rotated again, the first step port position is set again.

At the first step port position shown in FIG. 2A, only the first connection ports 10a, 20a and 30a of the rotary valves 10, 20, 30 and 40 are opened, and the second connection ports 20b, 30b and 40b, the third connection ports 30c, 40c and 10c, and the fourth connection ports 40d, 10d and 20d are closed.

At the first step port position, the desorbent port DP is connected to the first rotary valve 10, the extract port EP is connected to the second rotary valve 20, the feed port FP is connected to the third rotary valve 30, and the raffinate port RP is connected to the first rotary valve 10.

As a result, the desorbent injected from the desorbent port DP passes through the first rotary valve 10 and the first column 100 and is then injected into the second rotary valve 20.

The microalgae-derived multi-component mixture injected from the feed port FP is injected into the second rotary valve 20, is injected into the third rotary valve 30 together with the desorbent passing through the second column 200, and then passes through the third column 300.

In the present invention, the microalgae-derived multi-component mixture includes fucose, and includes monosaccharide components such as rhamnose, ribose, glucose, xylose, mannose and galactose. In addition, the microalgae-derived multi-component mixture of the present invention may further include amino acid components, such as alanine, glycine, proline, isoleucine and leucine, and a glycerol component. In the present invention, the other multi-component substance includes substances other than fucose in the microalgae-derived multi-component mixture.

An action of separating the mixture to be separated occurs due to the difference in the speed of progress between fucose and other multi-component ingredients after passing through the third column 300. Fucose is a slow-migrating component that moves slowly due to the strong adsorption force thereof, and other multi-component ingredients correspond to fast-migrating components that move rapidly due to the weak adsorption force thereof. While the first step port position is maintained, the fucose component may move through the fourth rotary valve 40 to the fourth column 400 but does not leave the fourth column.

Meanwhile, the other multi-component substance injected into the fourth rotary valve 40 passes through the fourth column 400 and is then injected into the first rotary valve 10 and discharged through the raffinate port RP.

Whenever a predetermined time (i.e., rotation time interval of the rotary valves) passes, the rotary valves 10, 20, 30 and 40 rotate to change the port position in the order of the second step port position (FIG. 2B), the third step port position (FIG. 2C), and the fourth step port position (FIG. 2D). During this sequential change of port position, the fucose, which is a slow-migrating component, shifts in the direction opposite to the port movement direction, eventually moving to the column near the extract port EP and being discharged through the extract port EP along the flow passage of the solvent. The criteria for the predetermined time mentioned above are described below.

In the present invention, an SMB process capable of continuously separating fucose, which is a high-valued rare sugar, at high purity and high yield, among the total of 7 types of monosaccharides (fucose, rhamnose, ribose, xylose, mannose, glucose and galactose) generated after the utilization of microalgae (N. oceanica) (extraction of biodiesel crude oil) has been developed. In order to reduce the development time and cost, and to guarantee the accuracy and ease of process scale-up and overall cost optimization, which are necessary in order to scale up to an industrial scale in the future, the process of developing a customized SMB process for fucose separation according to the present invention is based on a model-based design approach (approach using column model equations and parameters). First, adsorbents verified to have excellent separation selectivity and durability between fucose and other components among the aforementioned monosaccharide mixture components were screened. The result identified that a polydivinylbenzene-based hydrophobic adsorbent having a pore size of 100 Å satisfies all of the aforementioned requirements, and thus such a resin was selected as the adsorbent for the SMB process for fucose separation to be developed in the present invention. The multiple frontal analysis experiment was performed based on the selected adsorbent, and the intrinsic parameters (adsorption coefficient, size-exclusion factor and mass transfer coefficient) of monosaccharide components containing fucose were determined from the experiment data. An optimal design for the SMB process for fucose separation was performed according to the following procedure using the parameter values of each determined component and the latest genetic algorithm. First, the SMB process scheme, which is advantageous for improving the purity and yield of fucose, increasing the production concentration of fucose, reducing the costs of equipment and management, and improving operational robustness, was investigated. The result identified that a 3-zone open-loop model based on a 1-1-2 column configuration and port configuration in the order of desorbent→extract→feed→raffinate is an SMB scheme that satisfies all four requirements mentioned above. As a result, this scheme was chosen. The optimal operating conditions capable of maximizing the productivity of fucose while ensuring high purity and high yield of the fucose product under the selected scheme were determined. The theoretical verification of the customized fucose separation SMB process was performed under the optimal scheme and operating conditions determined according to this procedure. This verification was carried out using the result of a computer simulation for the column profile, and the result showed that all the solute bands have behaviors to accomplish the separation target. For experimental verification of the fucose separation SMB process that passed theoretical verification, SMB experiments to continuously separate only fucose from the model solution of the monosaccharide mixture were performed. As a result, the fucose product was continuously separated at a high purity of more than 97%, and no fucose loss occurred in this process.

Hereinafter, the present invention will be described in more detail with reference to examples. However, it will be obvious to those skilled in the art that these examples are provided only for illustration of the present invention and should not be construed as limiting the scope of the present invention.

Approach

1) Model-Based Design Approach

The following two requirements should be considered as priorities in the development of a new continuous separation process for a system. First, the development period and cost should be minimized. Second, optimal process operating conditions to maintain productivity and separation efficiency of the developed process at high levels should be determined. In order to satisfy both of these requirements, it is necessary to accurately understand the adsorption and mass transfer phenomena based on the column model and to identify various related parameter values. This approach to the process design based on column models and parameters is referred to as a “model-based design approach” (D. J. Wu et al., Ind. Eng. Chem. Res. 37 (1998) 4023-4035; P. H. Kim et al., J. Chromatogr. A 1406 (2015) 231-243). According to the present invention, an SMB process to conduct continuous separation of fucose was developed based on this approach.

2) Column-Model Simulation

One of the core steps in the model-based design approach is a process simulation using mathematical model equations. The mathematical model equations used in this step are transport phenomena equations that enable detailed prediction of the adsorption and mass transfer phenomena of each solute molecule in the column, which are often called “column model equations” (L. S. Pais et al., AIChE J. 44 (1998) 561-569; P. H. Kim et al., J. Chromatogr. A 1406 (2015) 231-243). Simulation refers to the process of calculating the solutions of this column model equation using a numerical method, which is often performed using a computer because a large amount of calculation is required for the calculation process.

There are many types of column model equations used for the simulation. Among them, the lumped mass-transfer model is adopted as a simulation model according to the present invention (Z. Ma et al., AIChE J. 43 (1997) 2488-2508; D. J. Wu et al., Ind. Eng. Chem. Res. 37 (1998) 4023-4035; P. H. Kim et al., J. Chromatogr. A 1406 (2015) 231-243). The reason is that this model is evaluated to be more accurate and efficient than other models. The adopted lumped mass-transfer model is depicted by the following equations.

$$\begin{matrix}{ϵ}_{b} \frac{\partial {C}_{b \text{?}}}{\partial \text{?} } + ( 1 - {ϵ}_{b} ) {K}_{\text{?}} ( \text{?} - \text{?} ) + \text{?} \text{?} \frac{\partial {C}_{b \text{?}}}{\partial z} - {ϵ}_{\text{?}} {E}_{\text{?}} = 0 & ( 1 a ) \\ {K}_{\text{?}} \text{?} \frac{\partial {C}_{i}^{*}}{\partial t} + ( 1 - {ϵ}_{\text{?}} ) \frac{\partial {q}_{i}}{\partial t} = {K}_{j , \text{?}} ( {C}_{\text{?}} - {C}_{\text{?}}^{\text{?}} ) \\ \text{?} \text{indicates text missing or illegible when filed} & ( 1 b )\end{matrix}$$

In the following equation, the subscript i represents solute, C_{b,i} and Cᵢ* represent the solute liquid concentrations in the inter-particle void (or mobile phase) and intra-particle void (or pore phase), respectively, and qᵢ represents the concentration in the adsorbent phase, which is in equilibrium with the liquid phase concentration in the pore phase. The case where the concentration of the liquid phase and the concentration of the adsorbent, which are in equilibrium with each other, follow a linear isothermal relationship can be expressed as the following linear isothermal model. In the following equation, Hᵢ refers to the linear isothermal parameter of solute i.

qᵢ=HᵢCᵢ² (2)

Meanwhile, in the column model equation above, K_{f,i} represents a lumped mass-transfer coefficient, and the value thereof can be calculated by the following method.

$$\frac{1}{{K}_{f , i}} = \frac{{( {d}_{p} / 2 )}^{2}}{15 {K}_{\text{?}} {ϵ}_{p} {D}_{p}} + \frac{( {d}_{p} / 2 )}{3 {k}_{f}} \\ \text{?} \text{indicates text missing or illegible when filed} \qquad ( 3 )$$

wherein dₚ represents a diameter of an adsorbent, and Dₚ and k_{f} represent an intra-particle diffusivity and a film mass-transfer coefficient, respectively.

The lumped mass-transfer model-based simulation described above is performed using an Aspen Chromatography simulator and is used for the measurement and verification of intrinsic parameters for the seven types of monosaccharide components mentioned in the previous section, the verification of the separation efficiency of the SMB process and the like. Furthermore, this model equation also plays a key role in the production of SMB optimization computational tools. Specific details regarding this section will be described below.

3) Production of SMB Optimization Tool

Another tool that plays a key role behind computer simulation in the model-based design approach is the SMB optimization computational tool. This tool is used to determine optimal operating conditions that satisfy the goals of the SMB process to be developed. The first requirement for the production of this optimization tool is an optimization algorithm. To date, stochastic theory-based genetic algorithms are known to be most effective in the optimization of multi-column counter-current mode processes such as SMB (R. B. Kasat et al., Comput. Chem. Eng. 27 (2003) 1785-1800; S. Mun et al., J. Chromatogr. A 1230 (2012) 100-109).

In the present invention, an SMB optimization computational program based on genetic algorithms was produced for the optimization of the fucose separation SMB process. Genetic algorithms have been developed several times recently. NSGA-II-JG (R. B. Kasat et al., Comput. Chem. Eng. 27 (2003) 1785-1800; S. Mun et al., J. Chromatogr. A 1230 (2012) 100-109), considered to be the latest genetic algorithm, was selected as a basic algorithm in the production of optimization tools according to the present invention.

Regarding the method of producing the SMB optimization tool, the optimization algorithm was coded using Visual Basic for Applications (VBA) programming language installed in Excel software. Based on this, NSGA-II-JG algorithm execution and column model simulation were performed simultaneously.

Preparation of Experiment

1) Materials

Seven types of monosaccharide components constituting the feed mixture were purchased from Sigma-Aldrich Co. All of water used in the experiment was distilled deionized water (DDW) which was obtained from a Milli-Q system purchased from Millipore (Bedford, Mass.). Sulfuric acid, used as a major component of the mobile phase in HPLC concentration analysis, was purchased from Yakuri Pure Chemicals Ltd. (Kyoto, Japan). The polydivinylbenzene-based hydrophobic resin (pore size=100 Å) finally selected as an adsorbent of the fucose invention was purchased from Purolite Co. (Philadelphia, Pa.). The average particle size of the adsorbent was 75 μm.

The adsorbent was charged into two different sized columns purchased from Bio-Chem Fluidics Co. (Boonton, N.J., US) before use. The sizes of the columns were 1.5×21.7 cm and 2.5×21.7 cm, respectively. Among them, the smaller column was used to test each candidate group in the step of selection of the adsorbent. The larger column was used to conduct the experiment to determine the intrinsic parameter of each monosaccharide component and the SMB experiment for continuous separation of fucose.

2) Instrumentation

Pulse Injection and Multiple Frontal Analysis Experimental Device

A Young-Lin HPLC system purchased from Young-Lin Instrument Corp. was used for pulse injection and multiple frontal analysis experiments. The system consists of a Young-Lin SP930D pump, a Young-Lin RI 750F detector and Autochro-3000 software. The Young-Lin SP930D pump is responsible for smooth transfer of solvents, while the Young-Lin RI 750F detector is responsible for real-time monitoring of each component concentration in the column effluent. The Autochro-3000 software is responsible for the control of the pumps and detectors and data collection.

SMB Process Device

The experimental device for the fucose separation SMB process according to the present invention was self-assembled and produced, was based on the 3-zone open-loop scheme as shown in FIG. 2, and had a column configuration of 1-1-2 and a port configuration of desorbent→extract→feed→raffinate. The reason for selecting this scheme will be described in detail with the invention result in the next section. The produced SMB device includes four rotary valves, four columns and three pumps. The rotary valve used for the SMB device is a select-trapping (ST) valve purchased from Valco Instrument Co. (Houston, Tex.). This valve connects each column to a corresponding port to maintain a flow configuration enabling continuous separation. The rotary valves were controlled using Labview 8.0 software. FIG. 2 shows the connection between the port and column in each step. Since a total of four columns are present, the port-column connection mode is continuously changed during four step changes, and after the step change is conducted four times, the subsequent port-column connection mode is returned again to the original mode. This changing of the port-column connection mode continues until the end of the SMB experiment. FIGS. 2A to 2D show the port-column connection mode in (a) an Nth step, (b) an (N+1)th step, (c) an (N+2)th step, and (d) an (N+3)th step. The flow of stream injected into the feed and desorbent ports of the SMB device was controlled using the Young-Lin SP 930D pump purchased from Young-Lin Instrument Corp., and the flow of stream discharged to the extract port was controlled using a Model QV pump purchased from Fluid Metering Inc. (Syosset, N.Y.). Meanwhile, the flow rate of the stream discharged to the raffinate port was determined using a mass balance without a separate pump.

HPLC Concentration Analyzer

A Waters HPLC system was used as an apparatus to analyze the concentrations of samples obtained by frontal experiment of the monosaccharide mixture and the SMB experiment for continuous separation of fucose. The solvent was transferred with a Waters 515 HPLC pump and concentration analysis of the samples was performed using a Waters 2414 RI detector. In addition, as a column for HPLC concentration analysis, a Bio-rad Aminex HPX-87H analytical column (0.78×30 cm) was purchased and used, and two analytical columns were connected in series and then used in order to increase the accuracy of concentration analysis. The injection of samples was performed using a Rheodyne 7725i injector and the volume of each injected sample was 5 μL. The mobile phase used for HPLC analysis was a 0.01M sulfuric acid solution and the flow rate was maintained at 0.4 mL/min. In addition, the temperature of the HPLC concentration analysis column was maintained at 65° C. using the Waters heater column module. The Waters HPLC system was controlled using Empower 2.0 software.

### Example 1: Selection of Adsorbent for Fucose Separation

1-1. Experiment Method

As a preliminary experiment for adsorbent selection, a 1.5×21.7 cm column filled with test subject adsorbents was installed in a Young-Lin HPLC system and pulse injection experiments were performed on each single monosaccharide component. The flow rate was maintained constant at 1 mL/min during the pulse injection experiment. The effluent history (concentration profile of stream discharged to the column outlet over time) data for each single monosaccharide component upon pulse injection was obtained in real time using an RI (refractive index) detector. The concentration of each monosaccharide component in the feed pulse was maintained at 20 g/L and the amount of the injected feed pulse was maintained at 200 μL.

1-2. Experimental Result

As a result of searching for commercial adsorbents that are expected to enable separation between monosaccharide components and are proven to be durable, the polydivinylbenzene-based hydrophobic adsorbent group was found to show the best performance. The polydivinylbenzene-based hydrophobic adsorbents applicable to monosaccharide separation can be classified into three kinds of resins according to pore size, and the physical properties of each resin are shown in Table 1. For convenience, these three types of resins are referred to as “adsorbent-a”, “adsorbent-b”, and “adsorbent-c”, respectively.

TABLE 1
Comparison in physical properties between polydivinylbenzene-
based hydrophobic adsorbents

| | Pore | Average | Surface | Bulk density |
|---|---|---|---|---|
| | diameter | particle | area | (g/mL of |
| | (Å) | diameter (μm) | (m²/g) | bed volume) |
| Adsorbent-a | 100 | 75 | >700 | 0.20~0.24 |
| Adsorbent-b | 200~300 | 75 | >600 | 0.17~0.20 |
| Adsorbent-c | 300~700 | 75 | >600 | 0.16~0.19 |

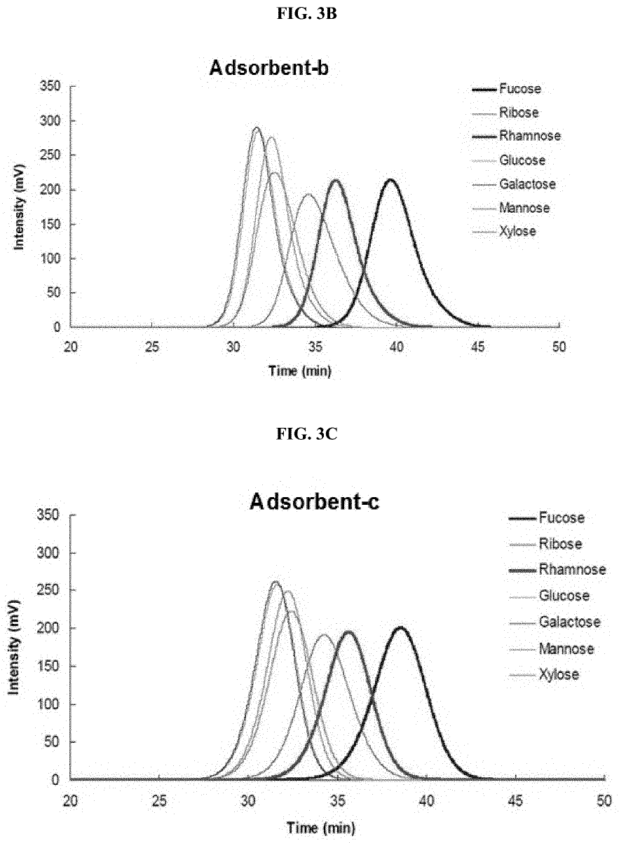

As shown in Table 1, the sizes of the adsorbents selected as candidate groups are all 75 μm. In consideration of the adsorbent particle size, all of the adsorbents set forth in Table 1 above are considered to be sufficiently applicable to large-scale chromatographic separation processes. In order to select the most suitable adsorbent for the fucose separation process from the three types of candidate adsorbents, pulse injection experiments were performed after filling a single column having a length of 21.7 cm and a diameter of 1.5 cm with each adsorbent candidate group. The results are shown in FIG. 3.

FIG. 3 shows the result of a pulse injection experiment (column dimensions: 1.5×21.7 cm, flow rate: 1 mL/min, injection volume: 0.2 mL) for the adsorbent candidate groups, and FIGS. 3A to 3C show the results of experiments for (a) adsorbent-a (pore size=100 Å), (b) adsorbent-b (pore size=250 Å), and (c) adsorbent-c (pore size=500 Å), respectively.

As can be seen from the result of pulse injection test of FIG. 3, the retention time of most monosaccharide components excluding rhamnose exhibited a significant difference from the retention time of fucose. This means that polydivinylbenzene-based hydrophobic adsorbents are suitable as adsorbents for the fucose separation process. However, since the rhamnose component disposed most adjacent to the fucose has the greatest influence on the separation of high-purity fucose, it is reasonable to determine the (separation) selectivity between the fucose and rhamnose components, and thereby select the adsorbent having the highest selectivity. The separation selectivity (α) between fucose and rhamnose was calculated according to the following equation (4).

$$α = \frac{{t}_{R 2} - {t}_{0}}{{t}_{R 1} - {t}_{0}} \qquad ( 4 )$$

wherein t_{R2} represents a residence time of fucose, t_{R1} represents a retention time of rhamnose, and t₀ represents a column void time. Table 2 shows the result of calculation of the separation selectivity according to Equation 4 above.

TABLE 2
Separation selectivity between fucose/rhamnose components
for polydivinylbenzene-based hydrophobic adsorbents

| | Adsorbent-a | Adsorbent-b | Adsorbent-c |
|---|---|---|---|
| | (pore | (pore | (pore |
| | size-100 Å) | size-250 Å) | size-500 Å) |
| Fucose/Rhamnose | 2.14 | 1.99 | 2.05 |
| (Separation | | | |
| selectivity(α) | | | |

Based on the result of Table 2, among the polydivinylbenzene-based hydrophobic resins, a resin having a pore size of 100 Å was selected as an adsorbent for the SMB process for fucose separation according to the present invention.

### Example 2: Measurement of Porosity of Adsorbent

2-1. Experimental Method

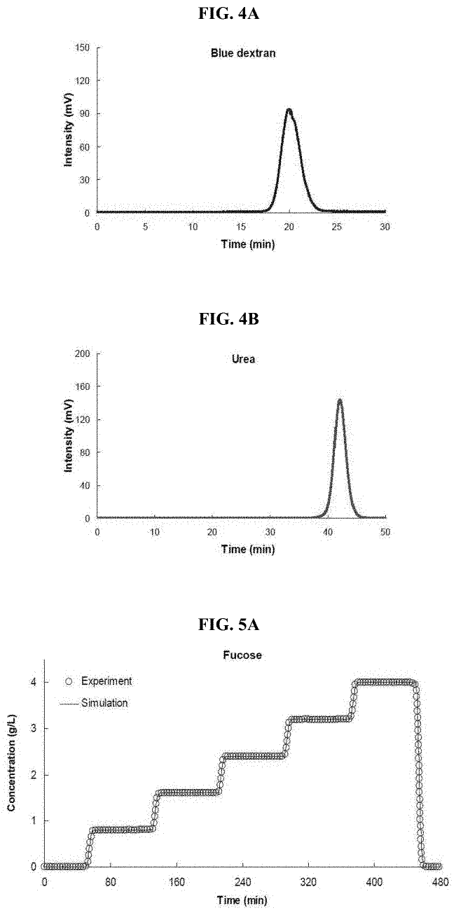

The porosity of the adsorbent (the polydivinylbenzene-based hydrophobic resin having a pore size of 100 Å) finally selected in Example 1 was measured. In the present example, an experiment to inject a tracer molecule having no adsorption property in a pulse form into a single column filled with the adsorbent was performed. The retention time can be measured from the concentration profile of the tracer molecule, obtained through the pulse injection experiment, and the porosity can be calculated from this data. Among porosities, the porosity between adsorbent particles, that is “bed voidage” was determined through pulse injection the experiment (FIG. 4A) on the blue dextran substance, and the porosity of the adsorbent particles, that is, “particle porosity”, was determined based on the result of the pulse injection experiment on the urea substance (FIG. 4B) and the results of measurement conducted before.

FIG. 4 shows the result of the tracer molecule pulse injection experiment (column dimension: 2.5×21.7 cm, flow rate: 2 mL/min, injection volume: 0.2 mL) for the finally selected adsorbent, and FIGS. 4A and 4B represent (A) blue dextran and (b) urea, respectively.

2-2. Experimental Result

As a result of measuring the porosity through the method described in 2-1 above, the porosity between the adsorbent particles, called “bed voidage (ε_{b})”, was 0.372, and the porosity of the adsorbent particles, called “particle porosity (εₚ)”, was 0.654.

### Example 3: Determination of Intrinsic Parameters of Each Monosaccharide Component-Adsorption Coefficient

3-1. Experimental Method

A multiple frontal analysis experiment was performed in order to obtain equilibrium adsorption data of each component in the chromatographic column, and the equilibrium between the adsorbent phase and the liquid phase in a column was maintained by continuously injecting a feed solution into the column (J. A. Vente et al., J. Chromatogr. A 1006 (2005) 72-79; Y. Xie et al., Ind. Eng. Chem. Res. 44 (2005) 6816-6823). At this time, in order to obtain equilibrium at several liquid concentrations, the concentration of the feed solution injected into the column was increased stepwise several times.

The column (2.5×21.7 cm) filled with the adsorbent finally selected in Example 1 was mounted on a Young-Lin HPLC system apparatus and then subjected to the multiple frontal analysis experiment described above. Two pumps and RI detectors were used in this experiment, and the device was controlled using Autochro-3000 software. Among the two pumps A and B used in the experiment, pump A was responsible for the delivery of DDW, and the other pump B was responsible for the delivery of each monosaccharide solution. The monosaccharide aqueous solution was continuously injected into the column until equilibrium between the adsorbent phase and the liquid phase in the column was achieved. Whether or not equilibrium is reached can be determined based on whether or not a concentration plateau occurs in the column effluent. When equilibrium is found to be reached, based on the occurrence of the concentration plateau, the concentration of the monosaccharide solution injected into the column was set to be higher than in the previous step, so that another equilibrium could be maintained in the column. The concentration of each monosaccharide component used in the experiment was maintained at 4 g/L, and the flow rate thereof was maintained at 2 mL/min. The concentration profile data of each component in the column effluent was collected through online monitoring using an RI detector. It is important that the flow of DDW and the monosaccharide solution (corresponding to the actual feed solution for the column) remain completely mixed before being injected into the column. For this purpose, the feed solution was passed through a mixer purchased from Analytical Scientific Instruments Co. immediately before being injected into the column.

3-2. Experiment Result

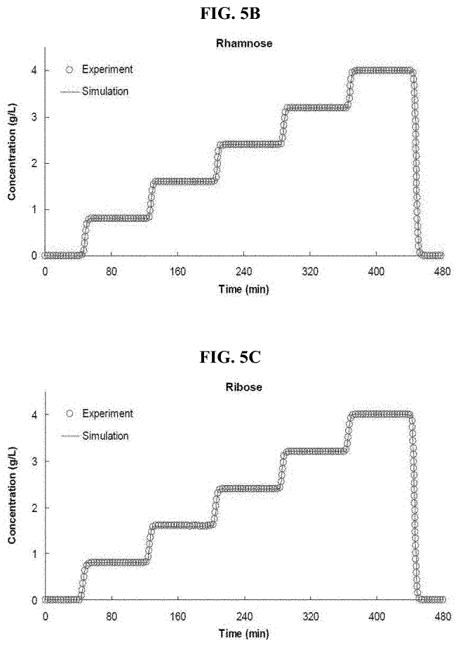

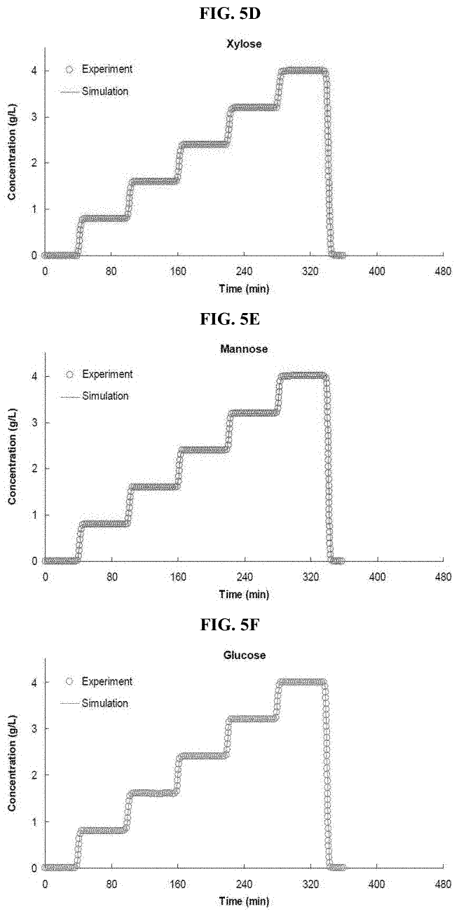

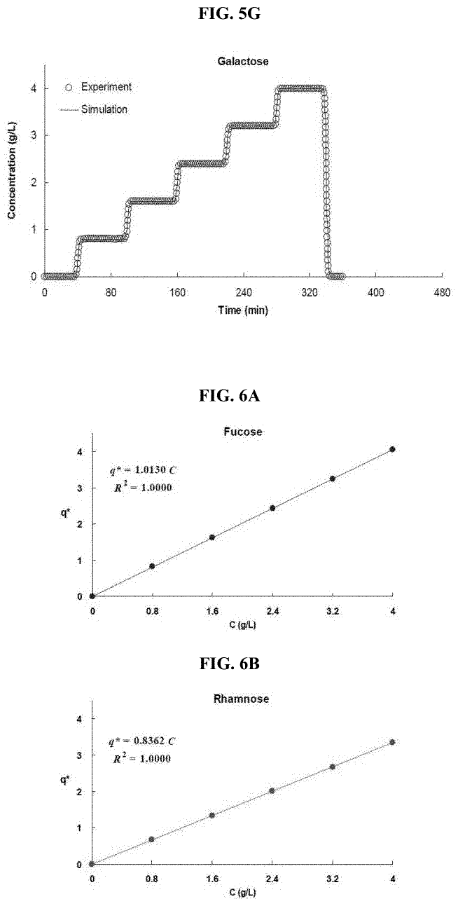

The adsorption coefficient of each monosaccharide component on the finally selected adsorbent was determined by the multiple frontal analysis method described in 3-1 above. The concentration of each monosaccharide component was set to 4 g/L during the multiple frontal analysis experiment, and this concentration corresponds to a set value covering the actual concentration range of each component in the monosaccharide mixture generated after pretreatment of defatted microalgal biomass. In addition, the flow rate was maintained at 2 mL/min. The length and diameter of the column used were 21.7 cm and 2.5 cm, respectively. The results of the multiple frontal analysis experiment performed on each monosaccharide component are shown in FIG. 5.

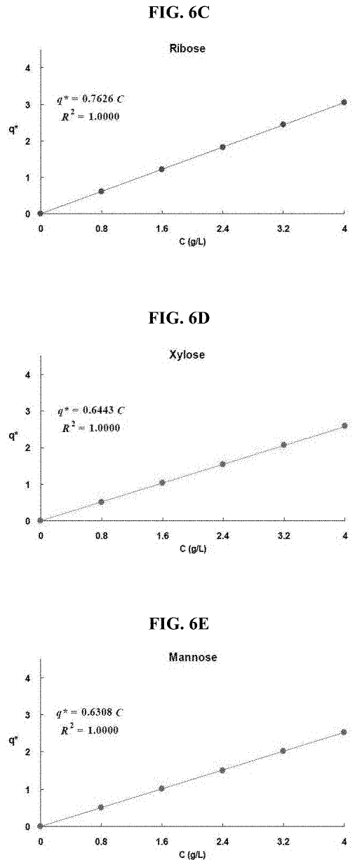

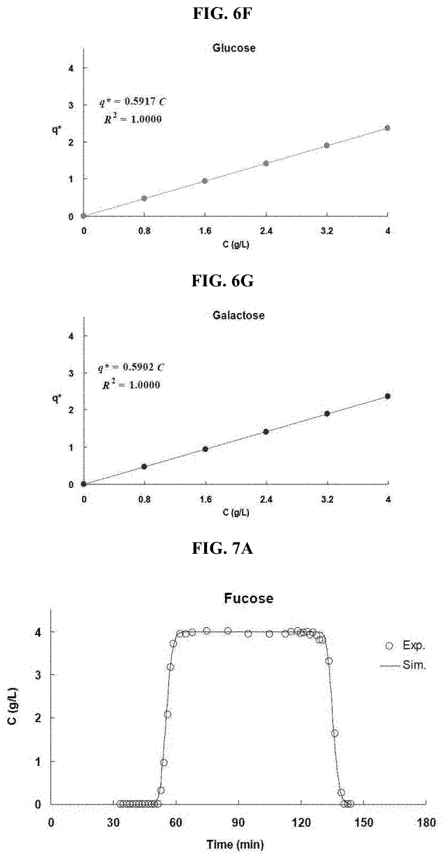

It can be seen in the concentration profile shown in FIGS. 5A to 5G that there is a concentration plateau region that remains the same as the feed concentration for a period of time. This is the region in which the solid and liquid phases in the column are in equilibrium and the concentration of the liquid phase at all positions in the column remains the same as the concentration of the feed injected into the column. For this reason, the equilibrium concentrations of liquid phases in solid-liquid equilibrium data, fall within the range of controllable variables that can be actually controlled. This is the advantage of the multiple frontal analysis method. Based on the results of FIG. 5 and the multiple frontal analysis induction equation, equilibrium capacity data (q* versus C) of each monosaccharide component on the adsorbent was obtained. The acquired equilibrium capacity data is shown in FIG. 6. As can be seen from FIG. 6, a linear relationship is formed between the equilibrium capacity (q*) data on each monosaccharide components and the liquid phase equilibrium concentration (C). The slope of this linear relationship corresponds to the retention factor (δ=(εₚKₑ+(1−εₚ)H) of each component. The retention factor of each component was calculated based on the (q*, C) data shown in FIG. 6. The result showed that the retention factor of fucose was the highest. The fact that fucose is not an intermediate retention-factor component in a monosaccharide mixture will serve as an advantage in future design of the fucose separation SMB process. In addition, it can be seen that there is a sufficient difference in retention factor between fucose and other monosaccharide components. These results indicate that the polydivinylbenzene-based hydrophobic resin (pore size=100 Å) adopted in the present invention is a very suitable adsorbent for the development of the fucose separation SMB process. Based on the retention factor results shown in FIG. 6, the linear isotherm parameter (H) and size-exclusion factor (Ke) of each monosaccharide component were calculated, and are shown in Table 3.

TABLE 3
Retention factor, linear adsorption coefficient (H), and size-
exclusion factor (Kₑ) of each monosaccharide on polydivinylbenzene-
based hydrophobic adsorbents (pore size = 100 Å)

| | Retention factor | Linear adsorption | Size-exclusion |
|---|---|---|---|
| | (δ) | coefficient (H) | factor (Kₑ) |
| Fucose | 1.0130 | 1.0376 | 1 |
| Rhamnose | 0.8362 | 0.5259 | 1 |
| Ribose | 0.7626 | 0.3130 | 1 |
| Xylose | 0.6442 | 0 | 0.985 |
| Mannose | 0.6308 | 0 | 0.965 |
| Glucose | 0.5917 | 0 | 0.905 |
| Galactose | 0.5902 | 0 | 0.902 |

### Example 4: Determination of Intrinsic Parameters of Each Monosaccharide Component-Mass Transfer Coefficient

For the design of the fucose separation SMB process, it is important to determine not only the adsorption coefficient of each monosaccharide component but also the mass transfer coefficients thereof. Mass transfer coefficients to be determined include an axial dispersion coefficient (E_{b}), a film mass-transfer coefficient (k_{f}), molecular diffusivity (D_{∞}) and intra-particle diffusivity (Dₚ). Among them, E_{b} and k_{f} are the mass transfer coefficients that depend on the linear velocity in the column as well as the properties of the material and liquid and solid phases, and the values thereof are determined mainly using correlations found in the literature. It is a common practice to specify the literature correlation that is used. In the present invention, Chung and Wen correlation (S F Chung et al., AIChE J. 14 (1968) 857-866) was used for E_{b}, and Wilson and Geankoplis correlation (E J Wilson et al., Ind. Eng. Chem. Fundam. 5 (1966) 9-14) was used for k_{f}. Meanwhile, D_{∞} and Dₚ, which are values of mass transfer coefficients that depend only on the properties of the material and liquid solid phases, and are independent of the linear velocity in the column. It is a common practice to generally report the values of D_{∞} and Dₚ. D_{∞} was calculated using a Wilke and Change correlation (C. R. Wilke et al., AIChE J. 1 (1955) 264-270). On the other hand, Dₚ was determined by obtaining the initial guess thereof from the Mackie and Mears correlation (J S Mackie et al., Proc. Roy. Soc. London Ser. A 232 (1955) 498-518) and correcting the value such that the concentration profile of the multiple frontal analysis experiment and simulation results are as close as possible. The determined values of D_{∞} and Dₚ of the monosaccharide components are shown in Table 4.

TABLE 4
Mass transfer coefficients of monosaccharide
components on polydivinylbenzene-based hydrophobic
adsorbents (pore size = 100 Å)

| | D_{∞} (cm²/min) | Dₚ (cm²/min) | E_{b} (cm²/min) | kᵢ (cm/min) |
|---|---|---|---|---|
| Fucose | 4.85 × 10⁻⁴ | 4.80 × 10⁻⁴ | Chung & | Wilson & |
| Rhamnose | 4.85 × 10⁻⁴ | 4.80 × 10⁻⁴ | Wen | Geankoplis |
| Ribose | 5.23 × 10⁻⁴ | 5.00 × 10⁻⁴ | corelation | correlation |
| Xylose | 5.28 × 10⁻⁴ | 5.00 × 10⁻⁴ | | |
| Mannose | 4.77 × 10⁻⁴ | 4.50 × 10⁻⁴ | | |
| Glucose | 4.77 × 10⁻⁴ | 4.50 × 10⁻⁴ | | |
| Galactose | 4.77 × 10⁻⁴ | 4.50 × 10⁻⁴ | | |

### Example 5: First Verification of Intrinsic Parameters-Computer Simulation

In order to verify the intrinsic parameters (adsorption coefficients, size-exclusion factors, mass transfer coefficients) of each monosaccharide component determined in Examples 3 and 4, a computer simulation was conducted based on the column model equation (Equation (1)), into which these parameter values were input. This task was performed using an Aspen Chromatography simulator, and the simulation conditions were kept the same as in the multiple frontal analysis experiment. The results of a comparison between the computer simulation results and the multiple frontal analysis data are shown in FIG. 5. In this drawing, the lines represent the simulation results and the symbols represent experiment data. As can be seen from FIG. 5, the simulation results and the multiple frontal analysis experiment data closely correspond to each other. This means that the values of adsorption coefficients, size-exclusion factors and mass transfer coefficients input during the simulation reflect the behavior of each monosaccharide component in the column.

### Example 6: Second Verification of Intrinsic Parameters-Mixture Injection Frontal Analysis Experiment

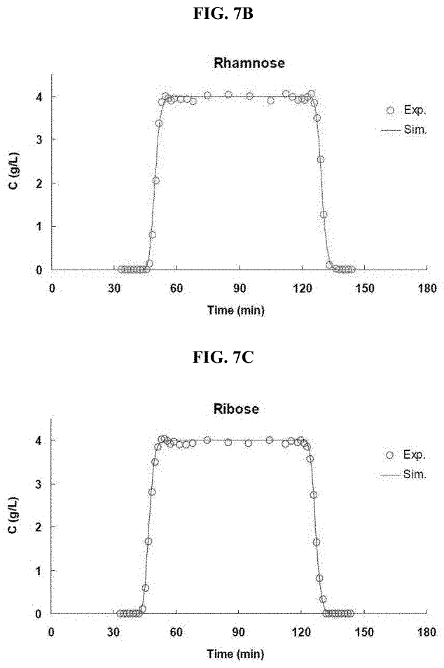

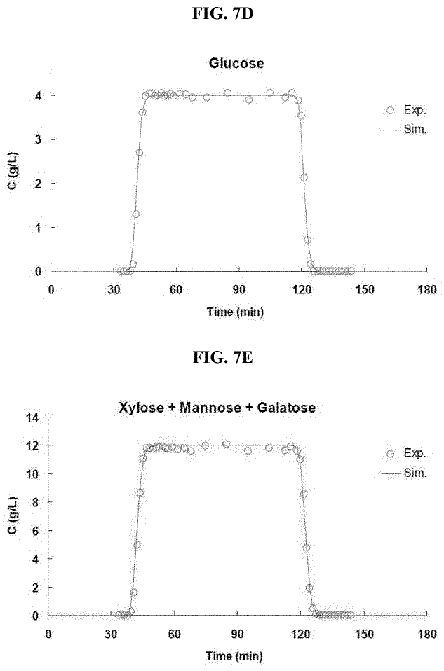

The verification results above were limited to the comparison of experiment data with simulation results for a single monosaccharide component. For further verification, a frontal analysis experiment, in which a monosaccharide mixture containing fucose was injected as a feed, (called a mixture frontal experiment) was performed (column dimension: 2.5×21.7 cm, flow rate: 2 mL/min, loading volume: 160 mL). In addition, simulations corresponding to these experimental conditions were performed based on the values of adsorption coefficients, size-exclusion factors and mass transfer coefficients determined in the previous step. A comparison between the mixture frontal experiment data of the monosaccharide mixture and the simulation results corresponding thereto is shown in FIG. 7. Due to the large number of mixture components, comparative data are presented for each component. Furthermore, in the case of xylose, mannose and galactose components, the peaks of the respective components overlap each other and the extinction coefficients (HPLC peak area per unit concentration) of these three components are almost the same. For this reason, these three components were subjected to integrated analysis instead of individual analysis.

As can be seen from the comparison result of FIG. 7, the mixture frontal experiment data for the monosaccharide mixture is predicted well by the corresponding simulation. It was confirmed that the mixture frontal experiment data for the monosaccharide mixture as well as the multiple frontal analysis experiment data for the monosaccharide single component were predicted well by the corresponding simulations. This confirms the validity of the values of adsorption coefficients, size-exclusion factors and mass transfer coefficients determined above, and furthermore, the values of these coefficients can be used as reliable foundational data in the design of a fucose separation SMB process.

### Example 7: Optimal Design of SMB Process for Continuous Fucose Separation

The optimal design of the SMB process for continuous separation of fucose from the monosaccharide mixture based on intrinsic parameters (adsorption coefficient, size-exclusion factors, mass transfer coefficients) of each fucose-containing monosaccharide component in Tables 3 and 4 was performed. As the first step of this invention, the basic schemes of the SMB process for fucose separation, that is, column configuration and port configuration, should be determined. Considerations for this step are as follows. First, the equipment and management costs of the SMB process should be minimized. Second, the operational robustness of the SMB process should be improved by adopting a simple pattern of operation, instead of a complex pattern of operation. Third, a configuration that maintains high purity and yield of fucose should be realized. Fourth, a configuration that maintains the product concentration of fucose at a high level should be realized. The configuration that satisfies the first and second ones among these four considerations is a 3-zone open-loop scheme, and the third consideration is solved by increasing the number of columns in the separation zone (two adjacent zones between feed ports). Finally, the fourth consideration is satisfied by establishing an enrichment zone for the fucose product so that the concentration of the fucose product can be maintained high. As shown in Table 3, since the retention factor of fucose is the largest among the monosaccharide mixture components, the fucose product is discharged through the extract port, and thus an enrichment zone for the extract product should be established.

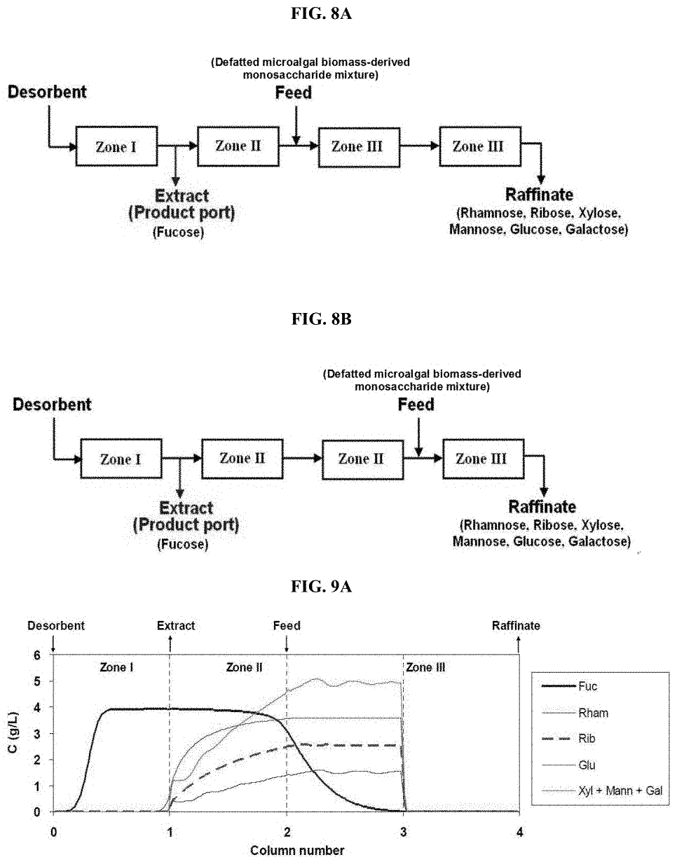

As a result of examining an SMB process scheme that can satisfy all four considerations described above, the two configurations shown in FIG. 8 (FIG. 8A shows a 3-zone open loop with a 1-1-2 column configuration, and FIG. 8B shows a 3-zone open loop with a 1-2-1 column configuration) were found to be most suitable. Both configurations are based on a 3-zone open-loop scheme and employ a port configuration in the order of desorbent→extract→feed→raffinate. However, the column configuration will be 1-1-2 or 1-2-1, depending on whether the zone in which an additional column will be disposed is zone II or zone III. The operating parameters (flow rates and port-switching time) were optimized for each of these two SMB process configurations (1-1-2, 1-2-1). This optimization was designed in order to maximize the throughput, which is directly related to SMB productivity and economic efficiency, and in the process, the constraint to maintain the purity and loss of fucose at 99% or more and less than 1%, respectively, was set. The optimization frame of the fucose separation SMB process including the constraint is presented below.

| | | |
|---|---|---|
| Max | J = Throughput [Q_{feed}, Q [symbol] , t [symbol] ] | (5a) |
| Subject to | Purity of fucose ≥ 99%. Loss of fucose ≤ 1% | (5b) |
| Fixed variables | Q_{des} = 5 mL/min | (5c) |
| | L [symbol] = 21.7 cm, d [symbol] = 2.5 cm | (5d) |
| | C_{feed} = 4 g/L for each organic acid | (5e) |
| | Extra-column dead volume = | (5f) |
| | 0.92% of bed volume | |
| Dependent | Q₁ (=Q_{des}) | (5g) |
| variables | Q₂ (=Q_{des} − Qₑₛₜ) | (5h) |
| | Q₃ (=Q_{des} − Qₑₛₜ + Q_{feed}) | (5i) |
| | Q_{raf} (=Q_{des} − Qₑₛₜ + Q_{feed}) | (5j) |

[symbol]

indicates data missing or illegible when filed

A SMB optimization computational tool based on the NSGA-II-JG algorithm (R. B. Kasat et al., Comput. Chem. Eng. 27 (2003) 1785-1800; S. Mun et al., J. Chromatogr. A 1230 (2012) 100-109) for optimization of the operating parameters of the fucose isolation SMB process presented above was produced. Optimization of each of two types of SMB configurations (1-1-2, 1-2-1) shown in FIG. 8 was performed using the produced tool and the results are shown in Table 5. As can be seen from Table 5, the optimal column configuration of 1-1-2 shows higher throughput than 1-2-1. Based on these results, 1-1-2 was finally selected as the column configuration of the fucose separation SMB process.

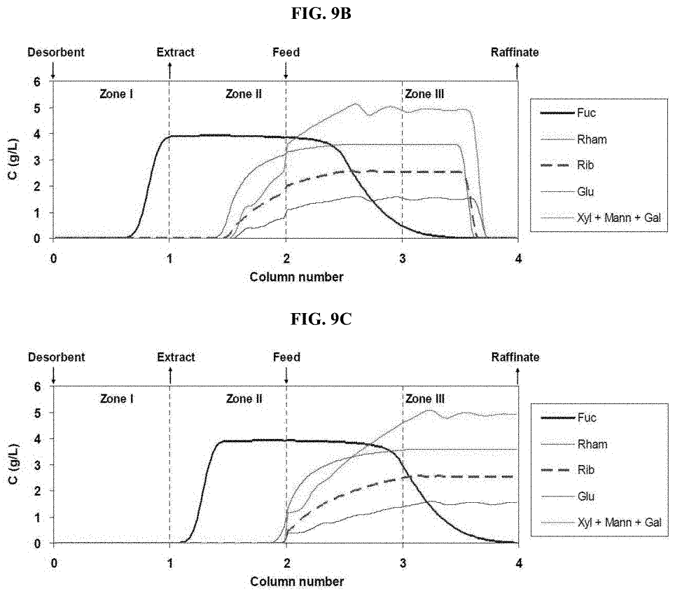

Computer simulations were performed based on the optimal operating parameters shown in Table 5 for theoretical verification of the result of the fucose separation SMB process optimization. As a result, a column profile was obtained in a cyclic steady state, and this is shown in FIG. 9. As can be seen from FIG. 9, the trailing wave and advancing wave of the fucose solute band are well confined in zone I and zone III. At the same time, it can be seen that the trailing waves of all monosaccharides other than fucose are well confined in zone II. The behaviors of each of these components in the SMB column can be a theoretical basis to guarantee continuous separation of fucose at high purity and high yield. FIGS. 9A to 9C show (a) the beginning of a switching period, (b) the middle of the switching period, and (c) the end of the switching period, respectively, and Fuc represents fucose, Rham represents rhamnose, Rib represents ribose, Glu represents glucose, Xyl represents xylose, Mann represents mannose, and Gal represents galactose.

TABLE 5
Results of optimization for fucose separation SMB process

| | 1-1-2 SMB | 1-2-1 SMB |
|---|---|---|
| | configuration | configuration |
| | (FIG. 8A) | (FIG. 8B) |
| Throughput (L/hr/100 L BV) | 6.62 | 5.49 |
| Q_{feed} (mL/min) | 0.47 | 0.39 |
| Q_{des} (mL/min) | 5.00 | 6.00 |
| Qₑₛₜ (mL/min) | 0.70 | 0.68 |
| Q_{raf} (mL/min) | 4.77 | 4.71 |
| t [symbol] (min) | 22.60 | 22.28 |

[symbol]

indicates data missing or illegible when filed

### Example 8: SMB Experiment

8-1. Experimental Method

As the first step of the continuous fucose separation experiment, the connection between each column and rotary valve and each pump in the SMB apparatus was performed as shown in FIG. 2. The SMB experiment starts from the operation of each pump and the implementation of LabVIEW 8.0 software. At the beginning of the experiment, a feed and a desorbent were continuously injected into the SMB. The feed solution was a mixture model solution containing 7 kinds of monosaccharides (fucose, rhamnose, ribose, xylose, mannose, glucose and galactose), and the concentration of each component was 4 g/L. Meanwhile, DDW was used as the desorbent. The SMB experiment was performed for up to 100 steps (over about 38 hours), the accuracy of the flow rate was checked at every step (switching period), and the concentrations of streams discharged from the extract and raffinate ports were analyzed in real time using the HPLC analysis system.

8-2. Experiment Result

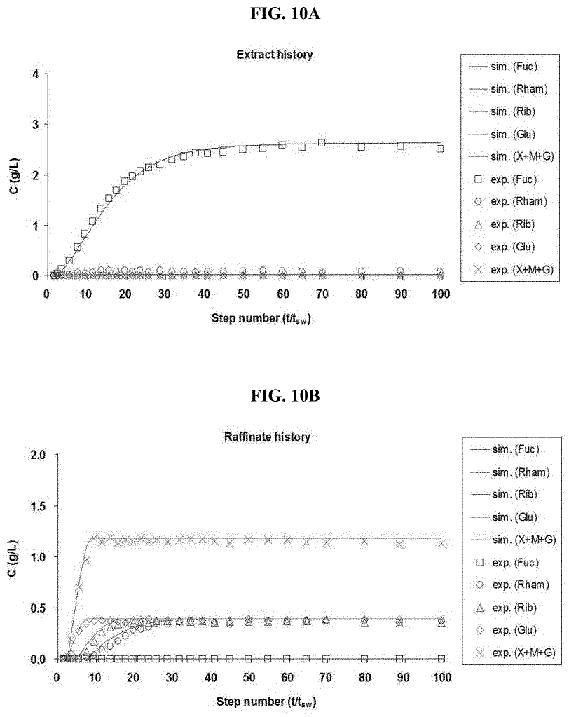

In order to experimentally verify the optimally designed fucose separation SMB process, the relevant SMB process experimental apparatus was assembled and produced. Based on the assembled SMB experimental apparatus and the optimum design results shown in Table 5, the continuous separation experiment for the fucose separation SMB process was performed for up to 100 steps (over 38 hours). Throughout the SMB experiment, a model solution including the entire defatted microalgal-biomass-derived monosaccharide component was continuously injected through the feed port. In addition, the streams continuously discharged through the extract and raffinate ports were collected. Concentration analysis was performed on all samples generated at that time, and the results are shown in FIG. 10. As can be seen from FIG. 10A, the content of components other than fucose in the stream discharged through the product port (extract port) of fucose, was very low. As a result, a fucose purity of 97.1% was obtained. Meanwhile, the experimental results of FIG. 10B showed that no fucose was lost through the impurity port (raffinate port) and that all of the components discharged through this port are monosaccharide components other than fucose. The concentration data of FIG. 10 corresponds to the average concentration for each step, and Fuc represents fucose, Rham represents rhamnose, Rib represents ribose, Glu represents glucose, and X+M+G represents xylose+mannose+galactose.

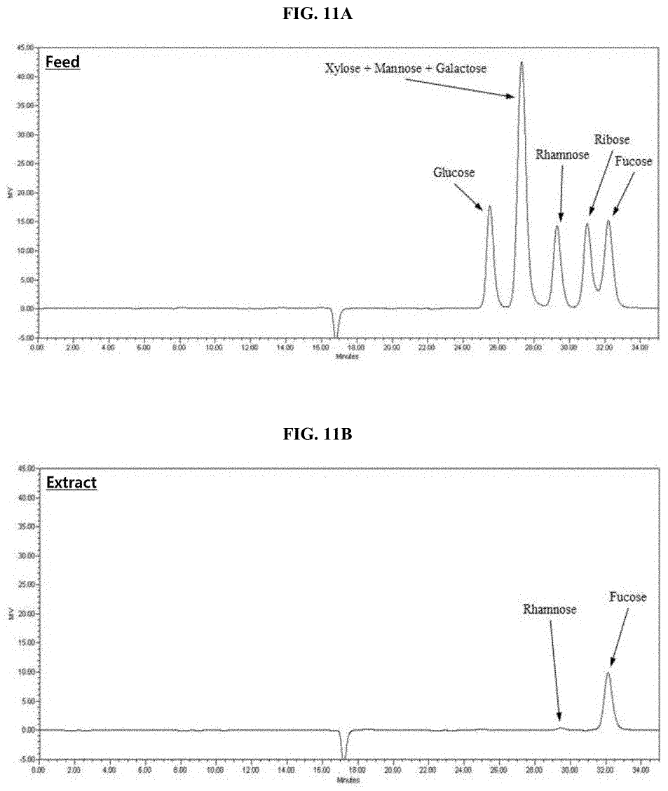

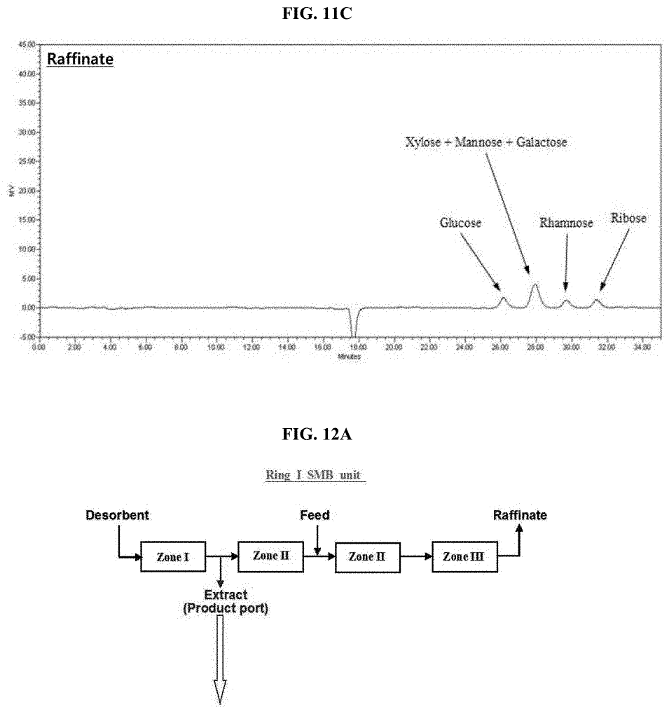

In addition, following the results of FIG. 10, additional experiment data demonstrating separation of fucose at high purity and high yield is shown in FIG. 11. FIG. 11A is an HPLC analysis chromatogram for the feed solution, and FIGS. 11B and 11C are HPLC analysis chromatograms for the extract and raffinate samples generated in the final step, respectively. As shown in FIG. 11B, only the fucose peak was clearly observed in the HPLC analysis chromatogram of the extract product, whereas the rhamnose peak was detected only in a very small amount, and no other monosaccharide peaks were detected. Meanwhile, in the HPLC analysis chromatogram of the raffinate (impurity) sample of FIG. 11C, only peaks of monosaccharide components other than fucose were identified, while no fucose peak was detected.

FIGS. 10 and 11 show that continuous separation of fucose at high purity and high yield from the defatted microalgal-biomass-derived monosaccharide mixture according to the present invention was successfully achieved. Furthermore, it can be seen that the result of computer simulation of the fucose separation SMB process developed in the present invention corresponds well to the SMB experiment data (FIG. 10). This means that the intrinsic parameter values of the monosaccharide components used for the optimization step of the fucose separation SMB process are appropriate, and that these parameter values can be fully utilized to realize optimal designs in future industrialization.

### Example 9: Additional SMB Experiment

In order to further expand the separation range of the SMB process described in the previous example, the SMB experiment of continuous separation of fucose was performed on the mixture containing additional components (amino acid substances and glycerol which may be produced together with monosaccharide substances after application of microalgae) other than monosaccharides (fucose, rhamnose, ribose, xylose, mannose, glucose and galactose).

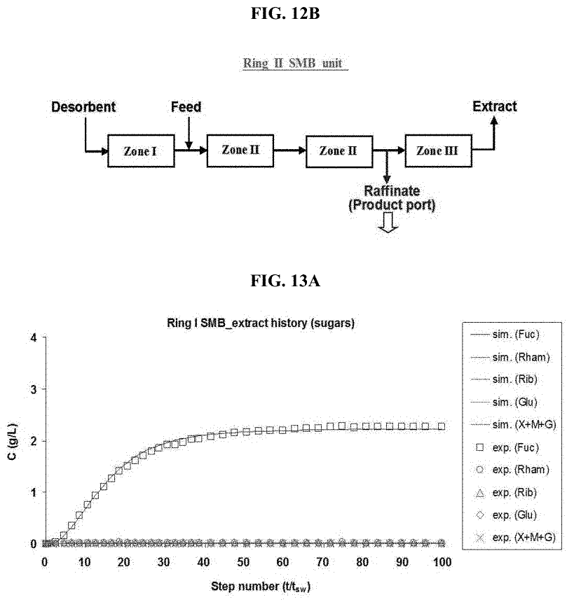

Prior to the SMB experiment mentioned above, the SMB process applicable to this embodiment was designed, and all of these processes were performed based on the procedure and approach of the previous embodiment. As the first step of the design process, multiple frontal analysis experiments were performed on the additional components (alanine, glycine, proline, isoleucine, leucine and glycerol) other than monosaccharides in order to determine the intrinsic parameters of each component. The result showed that the retention factors of isoleucine and leucine were higher than that of fucose, while the retention factors of the other components were lower than that of fucose. The optimal SMB process scheme for continuously separating fucose at high purity, reducing process equipment costs and improving process robustness was searched for based on the results of these multiple frontal analysis experiments. The result showed that it is most appropriate to use two SMB units of Ring I and Ring II, and to adopt the following column configuration and port configuration schemes (FIG. 12) for each SMB unit. The method for satisfying all three conditions mentioned above is that first, the Ring I SMB unit adopts the column configuration of 1-1-2 and the port configuration in the order of desorbent→extract→feed→raffinate (FIG. 12A), and then the Ring II SMB unit adopts the ring configuration of 1-2-1 and the port configuration (FIG. 12B) in the order of desorbent→feed→raffinate→extract. Under this SMB configuration, the Ring I unit functions to separate and remove rhamnose, ribose, xylose, mannose, glucose, galactose, alanine, glycine, proline and glycerol from fucose, and the Ring II unit functions to separate and remove isoleucine and leucine from fucose.

The experiment for continuous separation of fucose was performed based on the SMB process scheme and separation sequence described above. In this experiment, the feed solution injected into the feed port of the Ring I unit was a model solution containing 7 kinds of monosaccharides (fucose, rhamnose, ribose, xylose, mannose, glucose, galactose), 5 kinds of amino acids (alanine, glycine, proline, isoleucine, leucine), a glycerol component, and the like. The concentration of each component was set to 4 g/L. Meanwhile, the feed solution injected into the feed port of the Ring II unit was a mixture model solution containing fucose, isoleucine and leucine components. The concentration of each component was set to 4 g/L.

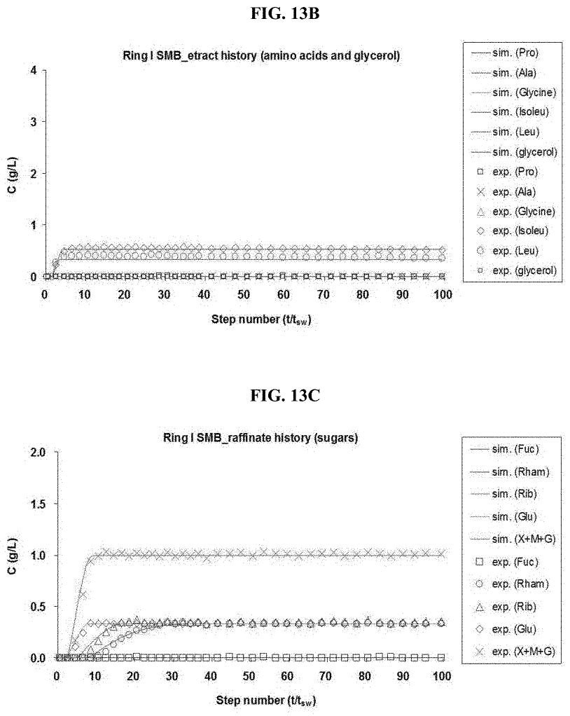

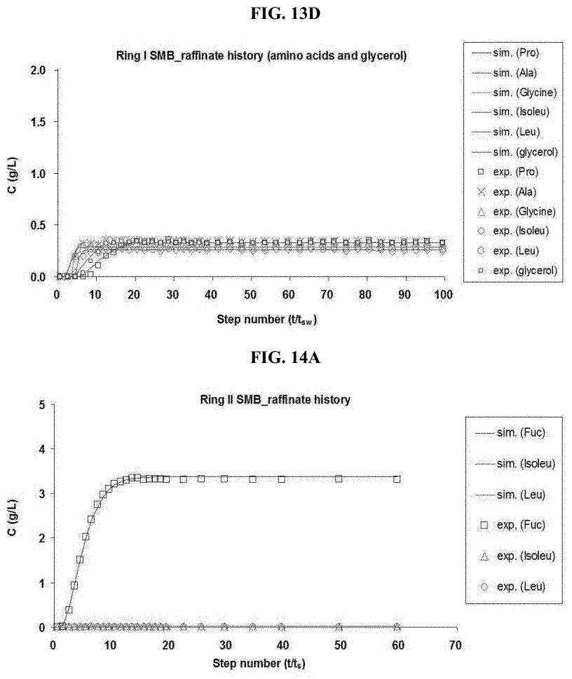

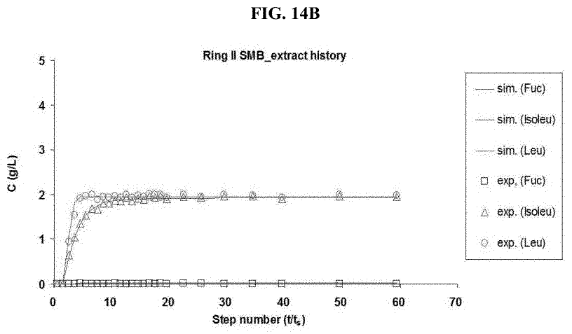

Ring I SMB and Ring II SMB experiment results are shown in FIGS. 13 and 14, respectively. As can be seen from FIG. 13, the components (rhamnose, ribose, xylose, mannose, glucose, galactose, alanine, glycine, proline, and glycerol) to be removed from the Ring I unit are almost all discharged through the raffinate port, and are not discharged through the extract port from which the fucose product is recovered. Furthermore, fucose products are also recovered only through the extract port, and are seldom discharged through the raffinate port. As a result, a fucose purity of 99.2% (purity based on the components to be removed of Ring I) was obtained in the Ring I SMB unit, and the fucose loss was low, specifically 0.9%. Meanwhile, as can be seen from FIG. 14, most of the components (isoleucine, leucine) to be removed from Ring II are discharged only through the extract port, and are seldom discharged through the raffinate port where the fucose product is recovered. In addition, fucose products are recovered only through the raffinate port and are seldom discharged through the extract port. As a result, in the Ring II SMB unit, a fucose purity of 99.5% was obtained and the fucose loss was low, that is, 0.5%.

The Ring I and Ring II SMB experiment results showed that the fucose separation method according to the present invention is capable of sufficiently securing continuous separation of fucose at high purity not only from a monosaccharide material generated after the use of microalgae, but also from a multi-component system including all other amino acid substances and glycerol.

Although specific configurations of the present invention have been described in detail, those skilled in the art will appreciate that this description is provided to set forth preferred embodiments for illustrative purposes and should not be construed as limiting the scope of the present invention. Therefore, the substantial scope of the present invention is defined by the accompanying claims and equivalents thereto.

### INDUSTRIAL APPLICABILITY

The fucose according to the present invention is separated from a microalgae-derived multi-component mixture using an SMB process, can be efficiently separated from various byproducts without using expensive solvents or reagents, and can be produced without causing problems associated with raw material supply costs and environmental damage for securing large quantities of fucose-containing wood. In addition, since the source of feed (raw materials) injected into the SMB process is derived from residual waste generated after the use of microalgae (lipid extraction), there are effects of minimizing the cost of securing raw materials and improving the economic efficiency of biodiesel production by microalgae. By using the SMB process of the present invention, it is possible to continuously separate fucose at high purity of 97% or more without any loss of fucose and to thereby dramatically increase the economic efficiency and industrial feasibility of fucose production.

1. A method of separating fucose based on SMB comprising:
injecting a desorbent into a desorbent port (DP);
recovering fucose from an extract port (EP);
injecting a microalgae-derived multi-component mixture into a feed port (FP); and
discharging other multi-component substance from a raffinate port (RP),
wherein fucose is separated using a porous polydivinylbenzene-based hydrophobic adsorbent in a plurality of columns connected to respective ports.

2. The method of separating fucose based on SMB of claim 1, wherein the hydrophobic adsorbent has a pore size of 50 to 900 Å.

3. The method of separating fucose based on SMB of claim 1, wherein the desorbent is water, a buffer, an acidic solution or a basic solution.

4. The method of separating fucose based on SMB of claim 1, wherein a purity of the fucose recovered from the extract port (EP) is 90% or more.

5. A device for separating fucose based on SMB using the method of claim 1, comprising:
a desorbent port (DP);
an extract port (EP);
a feed port (FP);
a raffinate port (RP);
a plurality of rotary valves 10, 20, 30 and 40 selectively connected to the ports (DP, EP, FP and RP), respectively; and
a plurality of columns 100, 200, 300 and 400 respectively provided in the plurality of rotary valves.

6. The device for separating fucose based on SMB of claim 5, wherein the plurality of rotary valves are connected to one another and are rotatable,
wherein the plurality of rotary valves each have a plurality of connection ports 10a, 10b, 10c, 10d, 20a, 20b, 20c, 20d, 30a, 30b, 30c, 30d, 40a, 40b, 40c, and 40d, and
wherein, as the rotary valves rotate, only one of the connection ports is opened and one of the rotary valves selectively connected to the ports DP, EP, FP and RP, respectively, is changed.

7. The device for separating fucose based on SMB of claim 5, wherein the rotary valves 10, 20, 30 and 40 are continuously changed according to rotation to cycle through a first step port position, a second step port position, a third step port position and a fourth step port position,
wherein the first connection port 10a, 20a, 30a is opened at the first step port position,
the second connection port 20b, 30b, 40b is opened at the second step port position,
the third connection port 30c, 40c, 10c is opened at the third step port position, and
the fourth connection port 40d, 10d, 20d is opened at the fourth step port position.

8. The device for separating fucose based on SMB of claim 7, wherein, at the first step port position,
the desorbent port (DP) is in fluid communication with a first column 100 through the first connection port 10a of a first rotary valve 10,
the extract port (EP) is in fluid communication with a first column 100 through the second connection port 20a of a second rotary valve 20,
the feed port (FP) is in fluid communication with a third column 300 and a fourth column 400 through the third connection port 30a of a third rotary valve 30, and
the fourth column 400 is in fluid communication with the raffinate port (RP) through the first connection port 10a of the first rotary valve 10.

9. The device for separating fucose based on SMB of claim 8, wherein a feed injected through the feed port (FP) at the first step port position is separated into fucose and other multi-component substance while passing through the third column 300 and the fourth column 400,
the fucose separated at the first step port position is shifted in a direction opposite to a port movement direction according to a sequential change of the port position, and is ultimately injected into the second rotary valve 20 and discharged from the extract port (EP), and
the other multi-component substance separated at the first step port position is injected into the first rotary valve 10 and is discharged from the raffinate port (RP).

10. The device for separating fucose based on SMB of claim 9, wherein when the rotary valves 10, 20, 30 and 40 are rotated from the first step port position to the second step port position, the feed injected through the feed port FP are separated into fucose and other multi-component substance while passing through the fourth column 400 and the first column 100,
the fucose separated at the second step port position is shifted in a direction opposite to the port movement direction according to the sequential change of the port position and is ultimately injected into the third rotary valve 30 and is discharged from the extract port (EP), and
the other multi-component substance separated at the second step port position is injected into the second rotary valve 20 and is discharged from the raffinate port (RP).

11. The device for separating fucose based on SMB of claim 5, wherein the plurality of rotary valves 10, 20, 30 and 40 are alternately changed in the first to fourth step port positions by rotating at every predetermined port-switching time, and at this time, time interval between rotation of the rotary valves is set so that the fucose and other multi-components are discharged to the extract port (EP) and the raffinate port (RP), respectively to change port position.
